(19) United States
(12) Patent Application Publication
(10) Pub. No.: US 2023/0021245 A1
(43) Pub. Date: Jan. 19, 2023

(54) Self-Sealing Membrane Connector for Gel Pads

(71) Applicant: C. R. Bard, Inc., Franklin Lakes, NJ (US)

(72) Inventors: Sean E. Walker, Platteville, CO (US); Audrey Earnshaw, Erie, CO (US); Angela Kontas, Denver, CO (US); Amy Lawrence, Port Orchard, WA (US); Ritaban Chakravarty, Louisville, CO (US); Brett R. Skelton, Louisville, CO (US); Eric D. Roberts, Arvada, CO (US)

(21) Appl. No.: 17/859,995

(22) Filed: Jul. 7, 2022

Related U.S. Application Data
(60) Provisional application No. 63/223,495, filed on Jul. 19, 2021.

(51) Int. Cl. A61F 7/02

(52) U.S. Cl. CPC A61F 7/02; A61F 2007/0054

(57) ABSTRACT

Disclosed herein are systems, methods, and a targeted temperature management (TTM) pad to facilitate thermal energy transfer between TTM fluid and a patient. The TTM pad includes a pad portion for placement on the patient and a fluid delivery conduit. The fluid delivery conduit can include a delivery conduit connector at a proximal end thereof and a fluid return conduit extending away from the pad portion. The fluid return conduit can include a return conduit connector at a proximal end thereof, and a connector coupled to a distal end of each of the fluid delivery conduit and the fluid return conduit. The connector can include a membrane configured to cover openings of both of the fluid delivery conduit and the fluid return conduit or includes multiple membranes, wherein the one or more membranes can include a piercing corresponding to each of the fluid delivery conduit and the fluid return conduit.

### PRIORITY

This application claims the benefit of priority to U.S. Provisional Application No. 63/223,495, filed Jul. 19, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. In general, TTM systems comprise a TTM fluid control module coupled with at least one thermal contact pad via a fluid delivery line. In some embodiments, tubing extends from a thermal contact pad to couple with the fluid delivery line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain thermally intimate pad-to-patient contact is of importance to fully realizing medical efficacies with TTM systems.

A fluid delivery line generally includes at least two fluid conduits for transporting TTM fluid to and from the thermal contact pad. Fluid delivery lines may include connection systems for selectively connecting to and disconnecting from the thermal contact pad. Although TTM systems may include a functionality to purge a thermal contact pad prior to disconnecting the thermal contact pad from a fluid delivery line, an operator may fail to utilize such functionality and, even when utilized, such functionality may leave some TTM fluid in the thermal contact pad. As a result, upon disconnection, some TTM fluid may leak from the tubing extending from the thermal connection pad thereby causing health and safety risks. Disclosed herein are systems, devices, and methods for preventing leakage of TTM fluid upon disconnecting a thermal contact pad from a fluid delivery line.

### SUMMARY OF THE INVENTION

Briefly summarized, disclosed herein is a targeted temperature management (TTM) system including a TTM module configured to provide a TTM fluid, a fluid delivery line (FDL) including a FDL hub, a fluid delivery lumen and a fluid return lumen and a pad configured to facilitate thermal energy transfer between the TTM fluid and a patient. The pad includes a pad portion configured for placement on the patient, a fluid delivery conduit extending away from the pad portion, the fluid delivery conduit including a delivery conduit connector at a proximal end thereof, a fluid return conduit extending away from the pad portion, the fluid return conduit including a return conduit connector at a proximal end thereof, and a connector coupled to a distal end of each of the fluid delivery conduit and the fluid return conduit, the connector including a first membrane covering an opening of the fluid delivery conduit and a second membrane covering an opening of the fluid return conduit.

Each of the first and second membranes are configured with a piercing, wherein the piercing of the first membrane is configured to receive a distal conduit tip of the fluid delivery lumen and the piercing of the second membrane is configured to receive a distal conduit tip of the return delivery lumen thereby establishing fluid communication between the FDL hub and the connector. The piercings may be formed as one of a circular piercing, a linear slit or a star piercing. The distal conduit tips of the fluid delivery lumen and the fluid return lumen may be tapered from a proximal point to a distal point. The connector includes a connector housing having disposed therein proximal ends of the fluid delivery conduit and the fluid return conduit, a conduit partition separates the fluid delivery conduit and fluid return conduit.

The connector includes a top compression strip connected to a top latch and a bottom compression strip connected to a bottom latch, wherein each of the top latch and bottom latch extends proximally from the connector. The top and bottom compression strips are configured to cause movement of the top and bottom latches in opposing directions upon application of pressure to the top and bottom compressions strips.

Also disclosed herein is a targeted temperature management (TTM) system including a TTM module configured to provide a TTM fluid, a fluid delivery line (FDL) including a FDL hub, a fluid delivery lumen and a fluid return lumen and a pad configured to facilitate thermal energy transfer between the TTM fluid and a patient. The pad includes a pad portion configured for placement on the patient, a fluid delivery conduit extending away from the pad portion, the fluid delivery conduit including a delivery conduit connector at a proximal end thereof, a fluid return conduit extending away from the pad portion, the fluid return conduit including a return conduit connector at a proximal end thereof, and a connector coupled to a distal end of each of the fluid delivery conduit and the fluid return conduit, the connector including a membrane configured to cover openings of each of the fluid delivery conduit and the fluid return conduit. The membrane is configured with a first piercing corresponding to an opening of the fluid delivery conduit and a second piercing corresponding to an opening of the fluid return conduit. The first and second piercings are formed as one of a circular piercing, a linear slit or a star piercing.

The first piercing is configured to receive a distal conduit tip of the fluid delivery lumen and the second piercing is configured to receive a distal conduit tip of the return delivery lumen thereby establishing fluid communication between the FDL hub and the connector. The distal conduit tips of the fluid delivery lumen and the fluid return lumen are tapered from a proximal point to a distal point. The connector includes a connector housing having disposed therein proximal ends of the fluid delivery conduit and the fluid return conduit, a conduit partition separates the fluid delivery conduit and fluid return conduit.

Additionally, a targeted temperature management (TTM) pad to receive and circulate TTM fluid to facilitate thermal energy transfer between the TTM fluid and a patient is disclosed where the TTM pad includes a pad portion configured for placement on the patient, a fluid delivery conduit extending away from the pad portion, the fluid delivery conduit including a delivery conduit connector at a proximal end thereof, a fluid return conduit extending away from the pad portion, the fluid return conduit including a return conduit connector at a proximal end thereof, and a connector coupled to a distal end of each of the fluid delivery conduit and the fluid return conduit, the connector including at least one membrane configured to cover at least an opening of one of the fluid delivery conduit or the fluid return conduit.

The connector includes the first membrane covering an opening of the fluid delivery conduit and a second membrane covering an opening of the fluid return conduit. Each of the first and second membranes are configured with a piercing, wherein the piercing of the first membrane is configured to receive a distal conduit tip of the fluid delivery lumen and the piercing of the second membrane is configured to receive a distal conduit tip of the return delivery lumen thereby establishing fluid communication between the FDL hub and the connector. The piercings may be formed as one of a circular piercing, a linear slit or a star piercing.

The distal conduit tips of the fluid delivery lumen and the fluid return lumen may be tapered from a proximal point to a distal point. The at least one membrane may be configured to cover openings of each of the fluid delivery conduit and the fluid return conduit, and wherein the membrane is configured with a first piercing corresponding to an opening of the fluid delivery conduit and a second piercing corresponding to an opening of the fluid return conduit.

The first and second piercings may be formed as one of a circular piercing, a linear slit or a star piercing, where the first piercing is configured to receive a distal conduit tip of the fluid delivery lumen and the second piercing is configured to receive a distal conduit tip of the return delivery lumen thereby establishing fluid communication between the FDL hub and the connector.

Also disclosed is a method of exchanging thermal energy with a patient, where the method includes providing a targeted temperature management (TTM) module configured to circulate TTM fluid through one or more pads, the TTM module including a fluid delivery line (FDL) for transporting the TTM fluid to and from the one or more pads, the FDL including an FDL hub, a fluid delivery lumen and a fluid return lumen, providing a pad, connecting the delivery conduit connector and the return conduit connector to the FDL hub to establish fluid communication of the fluid delivery conduit and the fluid return conduit with the FDL, applying the pad portion to the patient and initiating circulation of the TTM fluid through the pad. The pad includes a pad portion configured for placement on the patient, a fluid delivery conduit extending away from the pad portion, the fluid delivery conduit including a delivery conduit connector at a proximal end thereof, a fluid return conduit extending away from the pad portion, the fluid return conduit including a return conduit connector at a proximal end thereof, and a connector coupled to a distal end of each of the fluid delivery conduit and the fluid return conduit, the connector including at least one membrane configured to cover at least an opening of one of the fluid delivery conduit or the fluid return conduit.

The connector includes the first membrane covering an opening of the fluid delivery conduit and a second membrane covering an opening of the fluid return conduit. Each of the first and second membranes are configured with a piercing, wherein the piercing of the first membrane is configured to receive a distal conduit tip of the fluid delivery lumen and the piercing of the second membrane is configured to receive a distal conduit tip of the return delivery lumen thereby establishing fluid communication between the FDL hub and the connector.

The piercings may be formed as one of a circular piercing, a linear slit or a star piercing. The distal conduit tips of the fluid delivery lumen and the fluid return lumen may be tapered from a proximal point to a distal point. The at least one membrane is configured to cover openings of each of the fluid delivery conduit and the fluid return conduit, and wherein the membrane is configured with a first piercing corresponding to an opening of the fluid delivery conduit and a second piercing corresponding to an opening of the fluid return conduit.

The first and second piercings may be formed as one of a circular piercing, a linear slit or a star piercing. The first piercing is configured to receive a distal conduit tip of the fluid delivery lumen and the second piercing is configured to receive a distal conduit tip of the return delivery lumen thereby establishing fluid communication between the FDL hub and the connector. The connector includes a top compression strip connected to a top latch and a bottom compression strip connected to a bottom latch, wherein each of the top latch and bottom latch extends proximally from the connector. The top and bottom compression strips are configured to cause movement of the top and bottom latches in opposing directions upon application of pressure to the top and bottom compressions strips.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system for cooling or warming a patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of a thermal pad of the system of FIG. 1, in accordance with some embodiments.

FIG. 4B is a cross-sectional view of the pad of FIG. 4A cut along sectioning lines 4B-4B, in accordance with some embodiments.

FIG. 5A is a front view of a first embodiment of a pad connector of the system of FIG. 1, in accordance with some embodiments.

FIG. 5B is a side cross-sectional view of the pad connector of FIG. 5A, in accordance with some embodiments.

FIGS. 5C-5E are front views additional embodiments of a pad connector of the system of FIG. 1, in accordance with some embodiments.

FIG. 5F is a side cross-sectional view of the pad connector of FIG. 5F, in accordance with some embodiments.

FIG. 6A is a front view of a fluid delivery line hub of the system of FIG. 1, in accordance with some embodiments.

FIG. 6B is a side cross-sectional view of the fluid delivery line hub of FIG. 5A including a first embodiment of distal conduit tips, in accordance with some embodiments.

FIG. 6C is a side cross-sectional view of the fluid delivery line hub of FIG. 5A including a second embodiment of distal conduit tips, in accordance with some embodiments.

FIG. 7A is a view of a proximal portion of the pad connector of FIGS. 5A-5B approaching the fluid delivery line hub of the system of FIGS. 6A-6B, in accordance with some embodiments.

FIG. 7B is a view of the proximal portion of the pad connector and the fluid delivery line hub of FIGS. 6A-6B shown in a connected state, in accordance with some embodiments.

FIG. 7C illustrates the connected state of the proximal portion of the pad connector and the fluid delivery line hub of FIGS. 6A-6B having fluid flowing therethrough, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “horizontal,” “vertical” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled with” refer to any form of interaction between two or more entities, including mechanical, electrical, magnetic, electromagnetic, fluid, signal, communicative (including wireless), and thermal interaction. Two components may be connected to or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

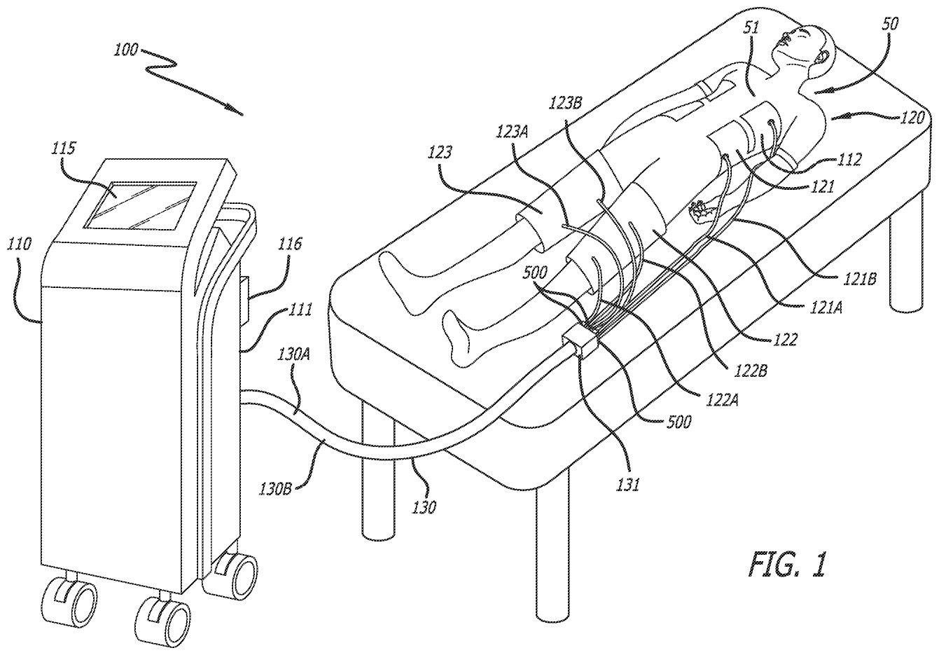

FIG. 1 illustrates a targeted temperature management (TTM) system 100 connected to a patient 50 for administering TTM therapy to the patient 50, where the therapy may include a cooling and/or warming of the patient 50, in accordance with some embodiments. The TTM system 100 includes a TTM module 110, a fluid delivery line (FDL) 130, and a thermal contact pad set 120. In the illustrated embodiment, the pad set 120 includes three thermal contact pads (pads) 121, 122, 123. In other embodiments, the pad set 120 may include one or more thermal contact pads (e.g., any number of pads). In the illustrated embodiments, the FDL 130 is configured to couple with two thermal pads. In other embodiments, the FDL 130 may be configured to couple with one or more thermal contact pads. In some embodiments, the system 100 may include more than one FDL 130.

Each pad includes a fluid delivery conduit and a fluid return conduit (sometimes referred to generally as the fluid conduits) coupled with the FDL 130 via an FDL hub 131. The FDL 130 includes a fluid delivery lumen 130A and a fluid return lumen 130B. In the illustrated embodiment, the pad 121 includes the fluid delivery conduit 121A coupled with the FDL 130 so as to be in fluid communication with the fluid delivery lumen 130A and a fluid return conduit 121B coupled with the FDL 130 so as to be in fluid communication with the fluid return lumen 130B. Similarly, the pad 122 includes the fluid delivery conduit 122A coupled with the FDL 130 so as to be in fluid communication with the fluid delivery lumen 130A and a fluid return conduit 122B coupled with the FDL 130 so as to be in fluid communication with the fluid return lumen 130B. Further, the pad 123 includes the fluid delivery conduit 123A coupled with the FDL 130 so as to be in fluid communication with the fluid delivery lumen 130A and a fluid return conduit 123B coupled with the FDL 130 so as to be in fluid communication with the fluid return lumen 130B. The proximal ends of the conduits 121A, 121B, the conduits 122A, 122B, and the conduits 123A, 123B may each terminate at a pad connector 500 discussed in detail below.

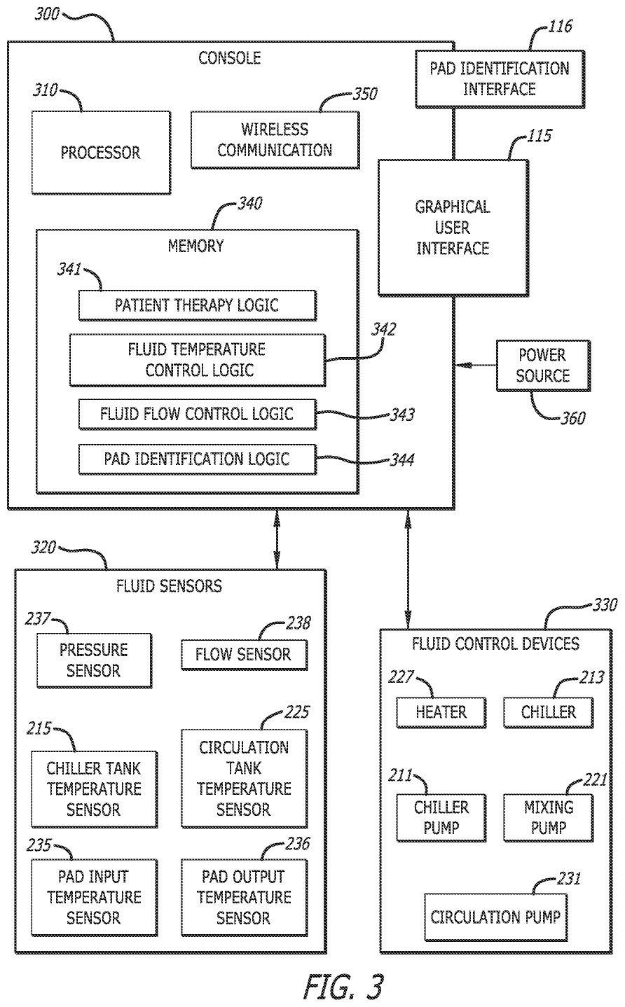

In use, the TTM module 110 prepares the TTM fluid 112 for delivery to the pad set 120 by heating or cooling the TTM fluid 112 to a defined temperature in accordance with prescribed TTM therapy parameters input by clinician via a graphical user interface 115. The TTM module 110 circulates the TTM fluid 112 between the TTM module 110 and the pad set 120 via the FDL 130. The pad set 120 is applied to the skin 51 of the patient to facilitate thermal energy exchange between the pad set 120 and the patient 50. During the TTM therapy, the TTM module 110 may continually control the temperature of the TTM fluid 112 toward a target temperature. The TTM module 110 may further include a pad identification interface 116 as further described below in relation to FIG. 3

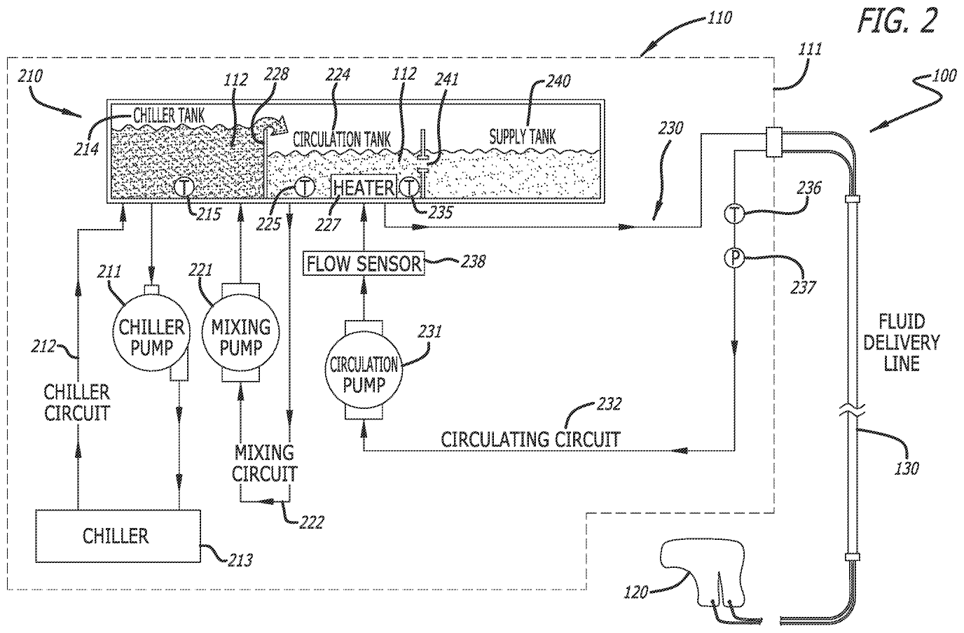

FIG. 2 illustrates a hydraulic schematic of the TTM system 100. The pad set 120 (FIG. 1) along with the corresponding fluid conduits are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may: 1) receive temperature data from the temperature sensor 215 within the chiller tank and the temperature sensor 225 within the circulation tank 224; and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 includes a circulation pump 213 to pull TTM fluid 112 from the circulation tank 224 and through a circulating circuit 232 that includes the pad set 120 located upstream of the circulation pump 213. The circulating circuit 232 also includes a pressure sensor 237 to represent a pressure of the TTM fluid 112 within the pad set 120. The circulating circuit 232 includes a temperature sensor 235 within the circulation tank 224 to represent the temperature of the TTM fluid 112 entering the pad set 120 and a temperature sensor 236 to represent the temperature of the TTM fluid exiting the pad set 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad set 120 so that a pressure within the pad set 120 is less than atmospheric pressure (i.e., negative) when TTM fluid flow through the circulating circuit 232 is stopped. The pad set 120 is also placed upstream of the circulation pump 231 to further establish a negative pressure within the pad set 120 when the circulation pump 213 is operating. The fluid flow control logic (see FIG. 3) may control the operation of the circulation pump 213 to establish and maintain a desired negative pressure within the pad set 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, fluid flow control logic 343, and pad identification logic 344. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled with the console 300 so that data from the fluid sensors 320 may be utilized in the performance of TTM module operations. Fluid control devices 330 are also illustrated in FIG. 3 as coupled with the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may include a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad set 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad set 120, the fluid flow control logic 343 may, in some embodiments, control the operation of the circulation pump 231 in accordance with a defined thermal energy exchange rate for the TTM therapy.

The console 300 may include or be coupled with a wireless communication module 350 to facilitate wireless communication with external devices. A power source 360 provides electrical power to the console 300.

The identification interface 116 may be coupled with the console 300 and provide pad identification data to the pad identification logic 344. The pad identification logic 344 may be configured so that, when executed by the processor 310, pad identification logic 344 may alert the clinician as to the identification of each thermal pad of the pad set 120. In an embodiment, the pad identification logic 344 may alert the clinician that one or more pads were not manufactured by a defined set of manufacturers. For example, if the identification interface 116 does not receive any pad identification data, the pad identification logic 344 may alert the clinician accordingly.

In some embodiments, the pad identification interface 116 may be configured to wirelessly receive pad identification data from the pad set 120. In the illustrated embodiment, the pad identification interface 116 may include a radio frequency identification (RFID) sensor configured to receive pad identification data from one or more RFID tags coupled with any or all pads of the pad set 120.

In some embodiments, the identification data may include a set of identification parameters (e.g., pad size), and the memory may include a corresponding set of identification parameters. An operation of the pad identification logic 344 may include comparing an identification parameter of the identification data with a corresponding identification parameter stored in memory, and the identification logic may be configured to modify the operation of the system in accordance with a result of the comparison.

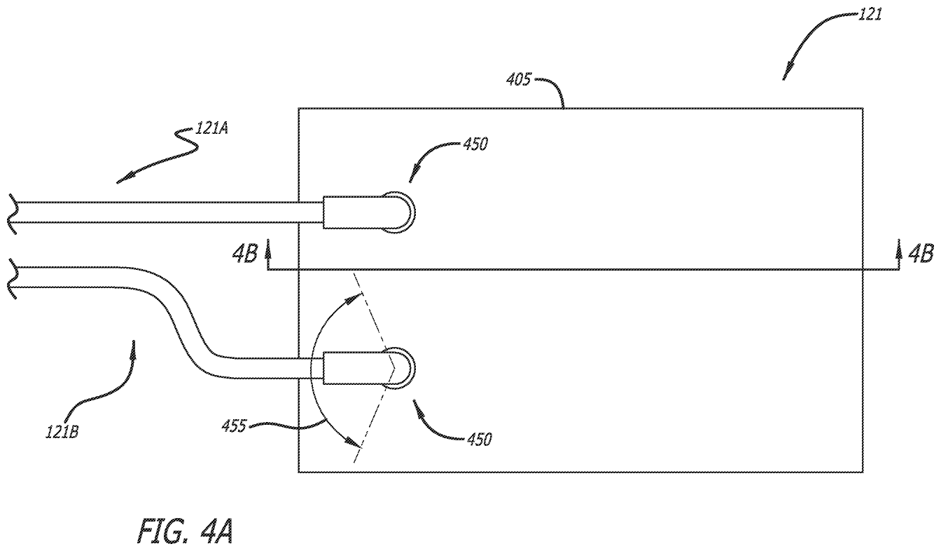

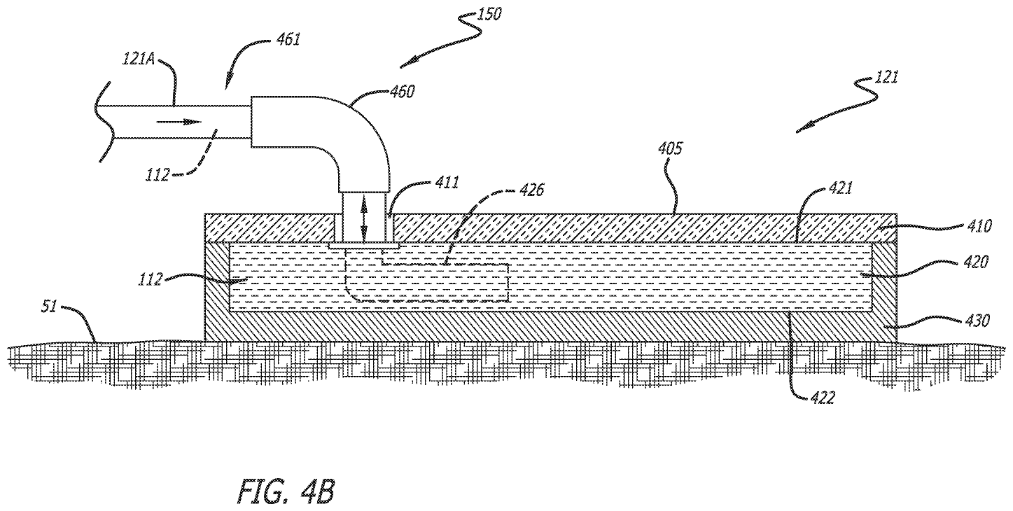

FIG. 4A shows a top view of the thermal contact pad 121. While the description that follows describes features, components and details of the pad 121, the description that follows may equally apply to any and all other thermal contact pads of the pad set 120. The fluid delivery conduit 121A and the fluid return conduit 121B extend away from the joints 450, in accordance with some embodiments. As illustrated, the joints 450 may provide for a rotatable connection between fluid delivery conduit 121A and the fluid return conduit 121B and a pad portion 405 of the pad 121. The rotatable connection may provide for the fluid conduit to rotate through an angle 455 ranging up to about 90 degrees, 180 degrees, 360 degrees, or more. In some embodiments, the joint 450 may define a fixed rotatable connection, i.e., the joint may allow rotation but not separation. In other embodiments, the joint 450 may define a pre-assembled rotatable connection that allows rotation and separation by the clinician.

FIG. 4B shows a cross-sectional side view of the pad portion 405 of the thermal contact pad 121 of FIG. 4A in contact with the patient 50, in accordance with some embodiments. The pad 121 may include multiple layers to provide multiple functions of the pad 121. A fluid containing layer 420 is fluidly coupled with the fluid delivery conduit 121A via the joint 450 to facilitate circulation of the TTM fluid 112 within the fluid containing layer 420. Similarly, (although not shown in FIG. 4B) the fluid containing layer 420 is fluidly coupled with the fluid return conduit 121B via the joint 450. The fluid containing layer 420 having TTM fluid 112 circulating therein defines a heat sink or a heat source for the patient 50 in accordance with a temperature of the TTM fluid 112. The fluid delivery conduit 121A may also be coupled with an internal fluid conduit 426 of the fluid containing layer 420 so that TTM fluid 112 entering the fluid containing layer 420 passes through the internal fluid conduit 426.

The pad 121 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the fluid containing layer 420 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence of space or air pockets between the thermal conduction layer 430 and the patient 50.

The pad 121 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may include one or more openings 411 extending through the insulation layer 410 to provide for coupling of the fluid delivery conduit 121A and fluid return conduit 121B with the fluid containing layer 420.

The joint 450 may include an elbow 460 to change the orientation of the fluid delivery conduit 121A. As shown, the orientation of 130 is shifted from an orientation that is perpendicular to the pad 121 to an orientation that is substantially parallel to the pad 121. The elbow 460 also establishes an orientation of a distal portion 461 of the fluid delivery conduit 121A to be substantially parallel to the pad 121 and/or the fluid containing layer 420.

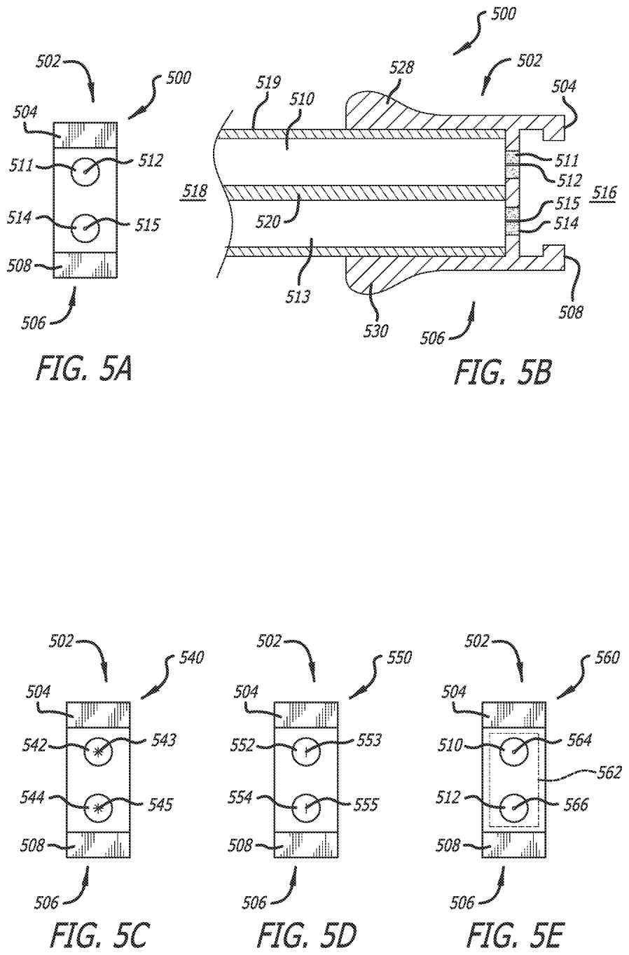

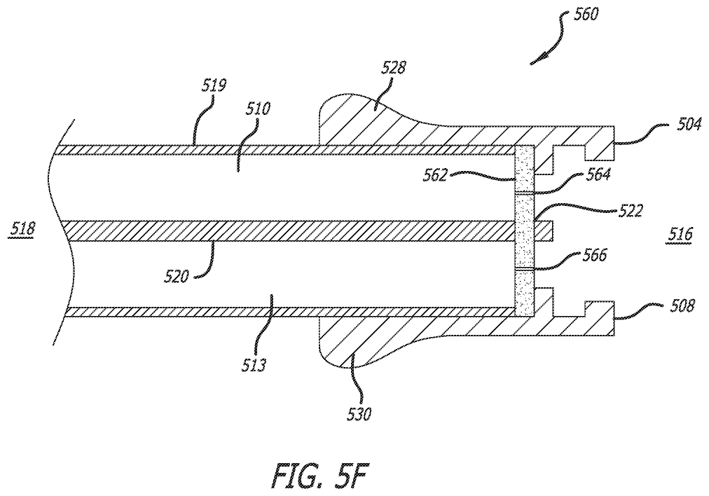

FIG. 5A is a front view of a first embodiment of the pad connector 500 of the system of FIG. 1, in accordance with some embodiments. The front view of the pad connector 500 illustrates a first (top) side 502 having a latch 504 and a second (bottom) side 506 having a latch 508. Further, the pad connector 500 includes one or more conduits 510, 513 (see FIG. 5B), where a membrane covers the opening of each conduit 510, 513. As illustrated, the membrane 511 covers the opening of the conduit 510 and the membrane 514 covers the opening of the conduit 513.

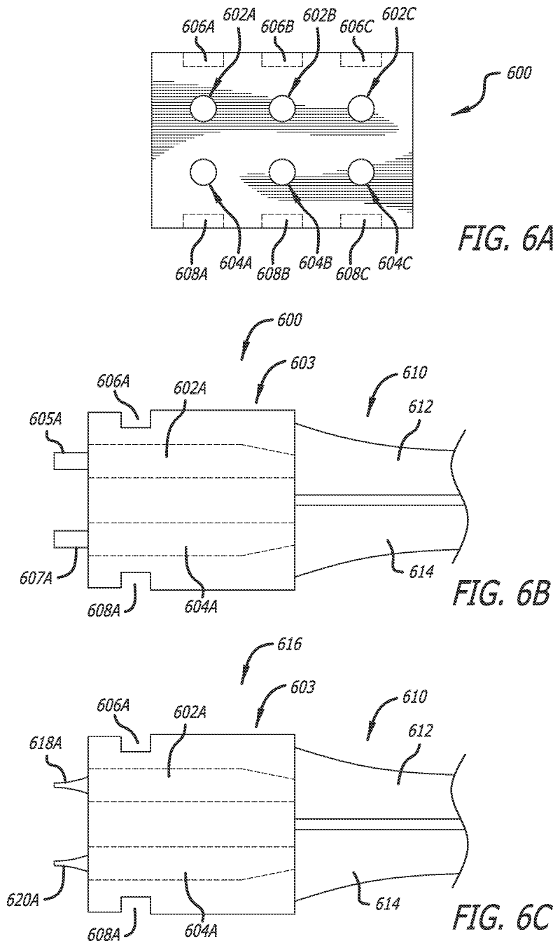

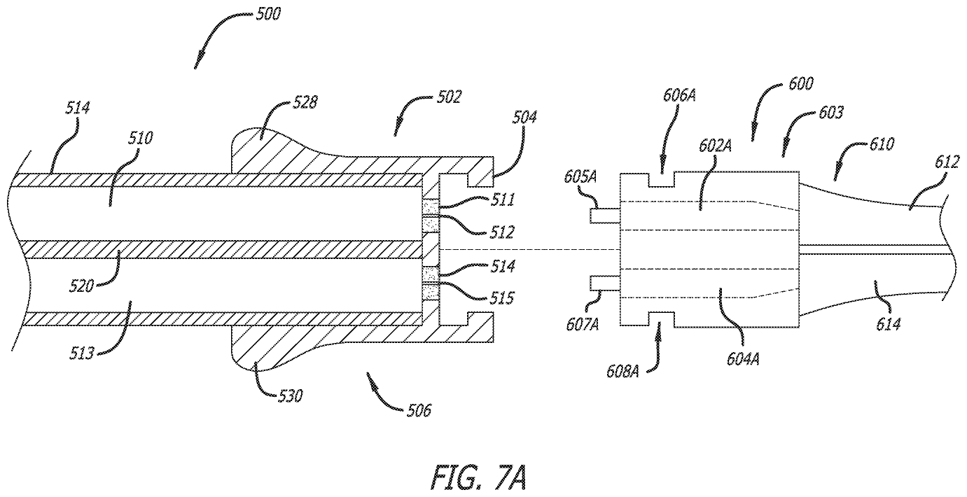

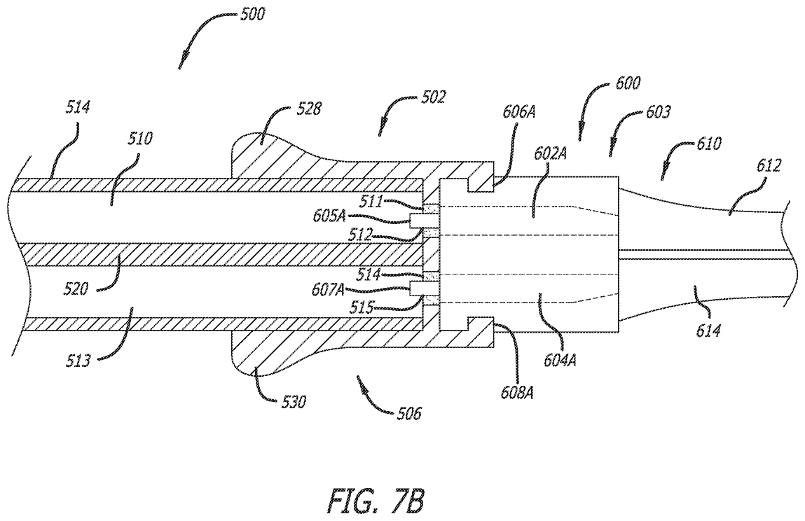

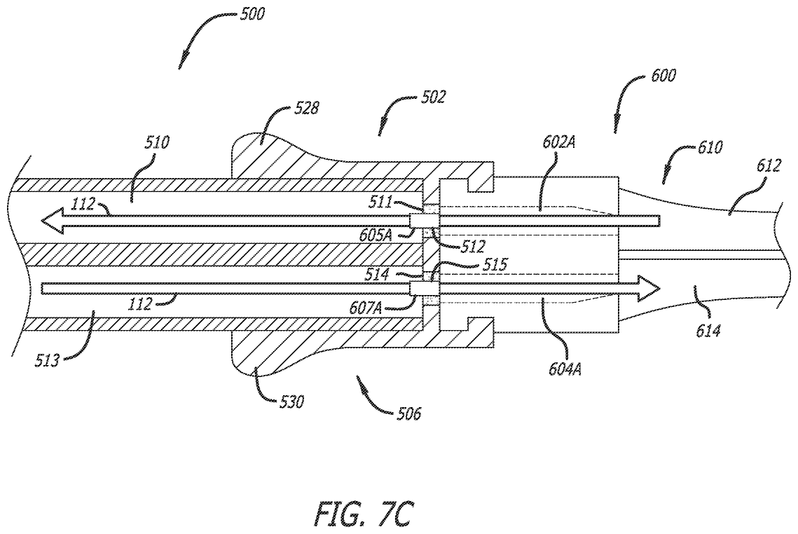

Conduit 510 may be configured to receive TTM fluid from a fluid delivery conduit (e.g., of the FDL hub 600 of FIGS. 6A-6B) and conduit 513 may be configured to return TTM fluid to a fluid return conduit (e.g., of the FDL hub 600 of FIGS. 6A-6B) when the membranes 511, 514 are pierced upon coupling of the connector 500 and the FDL hub 600 (see FIGS. 7A-7C). In some embodiments, the pad connector 500 may include a single conduit such that multiple pad connectors are utilized where a first such pad connector is configured to deliver TTM fluid and a second such pad connector is configured to return TTM fluid.

The membranes 511, 514 may be formed of suitable materials such as Chlorobutyl or other synthetic or natural elastic polymers. In some embodiments, the membranes 511, 514 may be non-latex and TEFLON®-coated. In some embodiments, the membranes 511, 514 may have a thickness of 1/16 inch, ⅛ inch, ¼ inch, etc.

The membrane 511 is configured with a piercing 512 that may be substantially located in the center of the membrane 511 and take various shapes. For example, FIG. 5A illustrates a circular piercing; however, FIGS. 5C-5D illustrate alternative piercings such as a star-pattern (e.g., a plurality of linear lines intersecting at a single point) (FIG. 5C) and a slit (FIG. 5D). The piercing 512 is configured to receive a distal conduit tip of the FDL 600. Referring again to FIG. 5A, the piercing 512 enables a distal conduit tip (e.g., the distal conduit tip 605A) to pass through the membrane 511 thereby establishing fluid communication between the pad connector 500 and the FDL hub 600. As the conduit 510 may represent a fluid delivery conduit and the distal conduit tip 605A may also correspond to a delivery fluid conduit 602A of the FDL hub 600, the fluid communication established therebetween enables TTM fluid to flow from the FDL hub 600 to the pad connector 500 and eventually to the pad 121.

The piercing 515 operates in the same manner as the piercing 512. Specifically, the piecing 515 operates to allow a distal conduit tip (e.g., the distal conduit tip 607A) to pass through the membrane 514 thereby establishing fluid communication between the fluid return conduit 513 of the pad connector 500 and the fluid return conduit 604A of the FDL hub 600.

Both membranes 511, 514 with their respective piercings operate to form a fluid seal across openings of their respective conduits when the pad connector 500 is de-coupled from the FDL hub 600. As a result, when the pad connector 500 is not coupled with the FDL hub 600, fluid is unable to pass through the membranes 511, 514. Thus, the membranes 511, 514 prevent leakage of TTM fluid that may remain in the pad 121 and/or its corresponding tubing (e.g., tubing 519) when the pad connector 500 is disconnected from the FDL hub 600. As noted above, even though a purging process may be performed prior to disconnecting the pad connector 500 and the FDL hub 600, there is no guarantee that such a process will be performed each time prior to disconnection and there is no guarantee that the purging process will remove all TTM fluid from the pad 121 and the tubing 519.

FIG. 5B is a side cross-sectional view of the pad connector of FIG. 5A, in accordance with some embodiments. The cross-sectional view of FIG. 5B illustrates that latches 504, 508 extends proximally from a proximal end 516 of the pad connector 500 (e.g., toward a TTM module such as the module 110 of FIG. 1) and that tubing 519 extends distally from a distal end 518 of from the pad connector 500 (e.g., toward a TTM pad such as the pad 121). FIG. 5B further illustrates that a conduit partition 520 is disposed within the connector 500 separating the conduits 510, 513.

FIG. 5B also illustrates that the pad connector 500 may include compression strips 528, 530, where application of pressure on the compression strips 528, 530 causes movement of the latches 504, 508 in opposing directions (e.g., away from a FDL hub 600) thereby allowing the pad connector 500 to connect and disconnect from the FDL hub 600. FIG. 5B also illustrates that the piercings 512, 515 extend the width of the membranes 511, 514 (which is also the case with respect to the piercings of FIGS. 5C-5D).

FIGS. 5C-5E are front views of additional embodiments of the pad connector 500 of FIGS. 5A-5B, in accordance with some embodiments. As noted above, the pad connector of 540 of FIG. 5C illustrates similar components at the pad connector 500 but includes membranes 542, 544 (similar to the membranes 511, 514) having piercings 543, 545 formed as a star-pattern, which may provide less resistance to an operator when a distal conduit tip of the FDL hub 600 is passing therefore during coupling of the pad connector 540 and the FDL hub 600 compared to when coupling of the pad connector 500 and the FDL hub 600. Additionally, the pad connector 550 of FIG. 5D illustrates similar components at the pad connector 500 but includes membranes 552, 554 (similar to the membranes 511, 514) having piercings 553, 555 formed as a slit, which may also provide less resistance during coupling than the piercings 512, 515 but slightly more resistance during coupling than the piercings 543, 545.

FIG. 5E provides yet another alternative embodiment of the pad connectors illustrated in FIGS. 5A-5D. Specifically, instead of individual membranes covering the openings of the conduits 510, 513, the pad connector 560 includes a single membrane 562 that spans and covers the openings of both conduits 510, 513. The membrane 562 is shown as having two piercings 564, 566, each located in substantially the center of the openings of the conduits 510, 513.

Referring to FIG. 5F, a side cross-sectional view of the pad connector of FIG. 5E is shown, in accordance with some embodiments. The cross-sectional view of the pad connector 560 illustrates numerous similar components as the pad connector 500 described above. However, different than the pad connector 500, the pad connector 560 includes the membrane 562 that spans and covers both openings of the conduits 510, 513, where, in such an embodiment, the conduit partition 520 may include an aperture 522 through which the membrane 562 may be disposed.

FIG. 6A is a front view of a fluid delivery line hub of the system of FIG. 1, in accordance with some embodiments. The FDL hub 600 may be one specific embodiment of the FDL hub 131 of FIG. 1. The embodiment of the FDL hub 600 illustrated in FIG. 6A includes a plurality of first fluid conduits 602A-602C and a plurality of second fluid conduits 604A-604C, where a pair of fluid conduits includes one of the first fluid conduits and a corresponding second fluid conduit, e.g., the conduits 602A, 604A or the conduits 602B, 604B for example. One conduit of a conduit pair may be configured to deliver TTM fluid to a first conduit of the pad connector 500 (e.g., the conduit 510) and the other conduit of the conduit pair may be configured to receive TTM fluid from a second conduit of the pad connector 500 (e.g., the conduit 513). For instance, the plurality of first fluid conduits 602A-602C may deliver TTM fluid to multiple instances of the pad connector 500 while the plurality of second fluid conduits 604A-604C may receive return TTM fluid from the multiple instances of the pad connector 500. It is noted that the number of conduits within each of the pluralities of the first and second fluid conduits 602A-602C, 604A-604C may differ from the illustration, which is not intended to be limiting. Additionally, the FDL hub 600 may include a pair of grooves for each conduit pair. As shown, a pair of grooves 606A, 608A corresponds to the conduit pair 602A, 604A, a pair of grooves 606B, 608B corresponds to the conduit pair 602B, 604B, and a pair of grooves 606C, 608C corresponds to the conduit pair 602C, 604C. Each pair of grooves assist in maintaining the coupling between the FDL hub 600 and an instance of the pad connector 500 (see FIGS. 7A-7C).

FIG. 6B is a side cross-sectional view of the fluid delivery line hub of FIG. 6A including a first embodiment of distal conduit tips, in accordance with some embodiments. The cross-sectional view of the FDL hub 600 illustrates a housing 603 houses the plurality of first fluid conduits 602A-602C and the plurality of second fluid conduits 604A-604C. The housing 603 includes two grooves 606A, 608A on opposing sides (e.g., on a top side and a bottom side). As is shown in FIGS. 7B-7C, the latches 504, 508 of the pad connector 500 are disposed within the grooves 606A, 608A when the pad connector 500 is connected to the FDL hub 600.

Additionally, the cross-sectional view of FIG. 6B illustrates that each conduit of both of the plurality of first fluid conduits 602A-602C and the plurality of second fluid conduits 604A-604C extend distally from the housing 603 (e.g., toward a TTM pad), where the distally extending portion may be referred to as distal conduit tips, where FIG. 6B provides a first embodiment of the distal conduit tips 605A, 607A. It should be understood that distal conduit tips also extend from first fluid conduits 602B-602C and second fluid conduits 604B-604C. Further, the cross-sectional view of FIG. 6B illustrates tubing 610 that extends proximally from the housing 603 (e.g., toward a TTM module such as the module 110 of FIG. 1), where the tubing 610 may be comprised of a delivery tubing 612 and a return tubing 614. In some embodiments, each of the plurality of first fluid conduits 602A-602C may receive TTM fluid from the delivery tubing 612 and each of the plurality of second fluid conduits 604A-604C may return TTM fluid to the return tubing 614.

FIG. 6C is a side cross-sectional view of a second embodiment of the fluid delivery line hub of FIG. 6A including a second embodiment of distal conduit tips, in accordance with some embodiments. The FDL hub 616 shares numerous components with the FDL hub 600, and thus may be a second embodiment of the FDL hub 131 of FIG. 1. Differently than the FDL hub 600, the FDL hub 616 includes an alternative embodiment of the distal conduit tips. Namely, the distal conduit tips 618A-620A extend distally from the housing 603 in a tapered manner. One operational advantage of the tapered distal conduit tips 618A-620A is that piercing the membranes of the pad connector 500 may be easier for an operator due to the narrower distal tip as compared to the distal conduit tips 605A, 607A of FIG. 6A. However, the FDL hub 616 couples with the pad connector 500 and otherwise operates in much the same manner as the FDL hub 600. Additionally, the FDL hub 616 may include multiple conduit pairs such as is shown with respect to the FDL hub 600 in FIG. 6A. More specifically, the FDL hub 616 may include conduit pairs such as the conduits 618A, 620A, conduits 618B, 620B (similar placement and operability as the conduits 602B, 604B), conduits 618C, 620C (similar placement and operability as the conduits 602C, 604C), etc.

FIG. 7A is a view of a proximal portion of the pad connector of FIGS. 5A-5B approaching a distal portion of the fluid delivery line hub 600 of FIGS. 6A-6B, in accordance with some embodiments. FIG. 7A illustrates the pad connector 500 and the FDL hub 600 prior to connecting (or immediately following disconnecting) using a combination of the cross-sectional illustrations of FIGS. 5B and 6B. As is shown, the latches 504, 508 align with the grooves 606A, 608A such that upon application of pressure to the compression strips 528, 530 of the pad connector 508, the latches 504, 508 will move in opposing directions allowing the pad connector 500 to physically connect with the FDL hub 600. As is understood, upon removal of the pressure from the compression strips 528, 530, the latches 504, 508 will return to a default position, which is within the grooves 606A, 608A when the pad connector 500 is connected with the FDL hub 600. Further, the conduit 602A aligns with the conduit 510 and the conduit 604A aligns with the conduit 513 thereby providing for fluid communication between the pad connector 500 and the FDL hub 600. Notably, as the illustration of FIG. 7A shows the pad connector 500 and the FDL hub 600 in a disconnected state, the membranes 511, 514 are shown in a closed position, e.g., preventing any fluid from passing through the openings of the conduits 510, 513.

FIG. 7B is a view of the pad connector 500 and the FDL hub 600 shown in a connected state, in accordance with some embodiments. As the pad connector 500 connects with the FDL hub 600, the distal conduit tips 605A, 607A enter the conduits 510, 513, respectively, while piercing the corresponding membrane 511, 514. Specifically, as the distal conduit tips 605A, 607A contact the membranes 511, 514, respectively, the distal conduit tip 605A passes through the membrane 511 via the piercing 512 and the distal conduit tip 607A passes through the membrane 514 via the piercing 515. As discussed above, such allows TTM fluid to flow between the conduits 602A, 510 and the conduits 604A, 513 (as seen in FIG. 7C).

FIG. 7C illustrates the connected state of the pad connector 500 and the FDL hub 600 having fluid flowing therethrough, in accordance with some embodiments. As shown, TTM fluid may flow from the conduit 602A (from a TTM module) to the conduit 510 and from the conduit 513 to the conduit 604A such that FIG. 7C illustrates fluid communication. As is understood, when the pad connector 500 is disconnected from the FDL hub 600 (upon application of pressure to the compression strips 528, 530 and opposing pulling forces applied to the pad connector 500 and the FDL hub 600), the distal conduit tips 605A, 607A are pulled from the membranes 511, 514, which return to their default (or non-deformed) state thereby once again blocking the openings of the conduits 510, 513 (as seen in FIG. 7A) and preventing TTM fluid to pass therethrough.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a fluid delivery line (FDL) including a FDL hub, a fluid delivery lumen and a fluid return lumen; and
a pad configured to facilitate thermal energy transfer between the TTM fluid and a patient, the pad comprising:
a pad portion configured for placement on the patient,
a fluid delivery conduit extending away from the pad portion, the fluid delivery conduit including a delivery conduit connector at a proximal end thereof,
a fluid return conduit extending away from the pad portion, the fluid return conduit including a return conduit connector at a proximal end thereof, and
a connector coupled to a distal end of each of the fluid delivery conduit and the fluid return conduit, the connector including a first membrane covering an opening of the fluid delivery conduit and a second membrane covering an opening of the fluid return conduit.

2. The system of claim 1, wherein each of the first and second membranes are configured with a piercing, wherein the piercing of the first membrane is configured to receive a distal conduit tip of the fluid delivery lumen and the piercing of the second membrane is configured to receive a distal conduit tip of the return delivery lumen thereby establishing fluid communication between the FDL hub and the connector.

3. The system of claim 2, wherein the piercings are formed as one of a circular piercing, a linear slit or a star piercing.

4. The system of claim 2, wherein the distal conduit tips of the fluid delivery lumen and the fluid return lumen are tapered from a proximal point to a distal point.

5. The system of claim 1, wherein the connector includes a connector housing having disposed therein proximal ends of the fluid delivery conduit and the fluid return conduit, a conduit partition separates the fluid delivery conduit and fluid return conduit.

6. The system of claim 1, wherein the connector includes a top compression strip connected to a top latch and a bottom compression strip connected to a bottom latch, wherein each of the top latch and bottom latch extends proximally from the connector.

7. The system of claim 6, wherein the top and bottom compression strips are configured to cause movement of the top and bottom latches in opposing directions upon application of pressure to the top and bottom compressions strips.

8. A targeted temperature management (TTM) system, comprising:
a TTM module configured to provide a TTM fluid;
a fluid delivery line (FDL) including a FDL hub, a fluid delivery lumen and a fluid return lumen; and
a pad configured to facilitate thermal energy transfer between the TTM fluid and a patient, the pad comprising:
a pad portion configured for placement on the patient,
a fluid delivery conduit extending away from the pad portion, the fluid delivery conduit including a delivery conduit connector at a proximal end thereof,
a fluid return conduit extending away from the pad portion, the fluid return conduit including a return conduit connector at a proximal end thereof, and
a connector coupled to a distal end of each of the fluid delivery conduit and the fluid return conduit, the connector including a membrane configured to cover openings of each of the fluid delivery conduit and the fluid return conduit.

9. The system of claim 8, wherein the membrane is configured with a first piercing corresponding to an opening of the fluid delivery conduit and a second piercing corresponding to an opening of the fluid return conduit.

10. The system of claim 9, wherein the first and second piercings are formed as one of a circular piercing, a linear slit or a star piercing.

11. The system of claim 9, wherein the first piercing is configured to receive a distal conduit tip of the fluid delivery lumen and the second piercing is configured to receive a distal conduit tip of the return delivery lumen thereby establishing fluid communication between the FDL hub and the connector.

12. The system of claim 11, wherein the distal conduit tips of the fluid delivery lumen and the fluid return lumen are tapered from a proximal point to a distal point.

13. The system of claim 8, wherein the connector includes a connector housing having disposed therein proximal ends of the fluid delivery conduit and the fluid return conduit, a conduit partition separates the fluid delivery conduit and fluid return conduit.

14. A targeted temperature management (TTM) pad to receive and circulate TTM fluid to facilitate thermal energy transfer between the TTM fluid and a patient, the TTM pad comprising:
a pad portion configured for placement on the patient,
a fluid delivery conduit extending away from the pad portion, the fluid delivery conduit including a delivery conduit connector at a proximal end thereof,
a fluid return conduit extending away from the pad portion, the fluid return conduit including a return conduit connector at a proximal end thereof, and
a connector coupled to a distal end of each of the fluid delivery conduit and the fluid return conduit, the connector including at least one membrane configured to cover at least an opening of one of the fluid delivery conduit or the fluid return conduit.

15. The TTM pad of claim 14, wherein the connector includes the first membrane covering an opening of the fluid delivery conduit and a second membrane covering an opening of the fluid return conduit.

16. The TTM pad of claim 15, wherein each of the first and second membranes are configured with a piercing, wherein the piercing of the first membrane is configured to receive a distal conduit tip of the fluid delivery lumen and the piercing of the second membrane is configured to receive a distal conduit tip of the return delivery lumen thereby establishing fluid communication between the FDL hub and the connector.

17. The TTM pad of claim 16, wherein the piercings are formed as one of a circular piercing, a linear slit or a star piercing.

18. The TTM pad of claim 16, wherein the distal conduit tips of the fluid delivery lumen and the fluid return lumen are tapered from a proximal point to a distal point.

19. The TTM pad of claim 14, wherein the at least one membrane is configured to cover openings of each of the fluid delivery conduit and the fluid return conduit, and wherein the membrane is configured with a first piercing corresponding to an opening of the fluid delivery conduit and a second piercing corresponding to an opening of the fluid return conduit.

20. The TTM pad of claim 19, wherein the first and second piercings are formed as one of a circular piercing, a linear slit or a star piercing.

21. The TTM pad of claim 19, wherein the first piercing is configured to receive a distal conduit tip of the fluid delivery lumen and the second piercing is configured to receive a distal conduit tip of the return delivery lumen thereby establishing fluid communication between the FDL hub and the connector.

22-31. (canceled)
